(19) United States
(12) Patent Application Publication
(10) Pub. No.: US 2025/0221746 A1
(43) Pub. Date: Jul. 10, 2025

(54) ROTATIONALLY STABLE TELESCOPING INTRAMEDULLARY NAIL

(71) Applicant: Imed Zavani, Dollars-Des-Ormeaux (CA)

(72) Inventor: Imed Zavani, Dollars-Des-Ormeaux (CA)

(73) Assignee: Imed Zavani, Dollars-Des-Ormeaux (CA)

(21) Appl. No.: 18/406,182

(22) Filed: Jan. 7, 2024

(51) Int. Cl. A61B 17/72; A61B 17/00; A61B 17/56

(52) U.S. Cl. CPC A61B 17/7283; A61B 2017/00526; A61B 2017/00991; A61B 2017/564

(57) ABSTRACT

A telescopic nail with rotational stability, where the female component is externally D-Shaped through a special burnishing process or a plastic deformation, and the male component nests in the form of the female. The male component is partially D-Shaped to lock the two components together for easy removal. The thread of the tapered head of the female component has multi-leads and is synched with the multi-lead thread of the proximal head on the male components. The number of leads and the length of the thread are adaptable to the child's bone anatomy in such ay there is no intrusion of the bone joint. The male and female are locked together as an assembled nail and inserted simultaneously in the bone intramedullary canal. In case of revision or hardware removal surgeries, the assembly nail components are removed simultaneously together.

### REFERENCES

Dubow H I., B. R. (1963). Studies of longitudinal bone growth resulting in an extensible nail. Surgical Forum, 14:455-8.
Francois Fassier, P. D. (2003). U.S. Pat. No. 6,524,313 B1.
Inan, A. F. (2019). U.S. Pat. No. 10,433,886B2.
Niemann. (1981). Surgical treatment of the tibia in osteogenesis imperfecta. Clin Orthop Relat Res., 159:134-40.
Oliver Birke, N. D. (2011). Experience With the Fassier-Duval Telescopic Rod: First 24 Consecutive Cases With a Minimum of 1-Year Follow-up. J Pediatr Orthop, 31(4):458-64.
Patch, E. J. (1977). U.S. Pat. No. 4,016,874.
Sofield H A, M. E. (1959). Fragmentation, realignment, and intramedullary rod fixation of deformities of the long bones in children: a ten-year appraisal. J Bone Joint Surg Am, 1371-1391.
Stockley I, B. M. (1989). The role of expanding intramedullary rods in osteogenesis imperfecta. J Bone Joint Surg Br., 71:422-427.
Tae-Joon Cho, I. H. (2007). Interlocking Telescopic Rod for Patients with Osteogenesis Imperfecta. J Bone Joint Surg Am, 89(5):1028-35.
Wilkinson J M, S. B. (1998). Surgical stabilization of the lower limb in osteogenesis imperfecta using the Sheffield Telescopic Intramedullary Rod System. J Bone Joint Surg Br., 80:999-1004.
Zeitlin L, F. F. (2003). Modern approach to children with osteogenesis imperfecta. J Pediatr Orthop B., 12:77-87.

### FIELD

The present disclosure pertains generally to a telescoping intramedullary nail with rotational stability. The nail described in this patent comprises two components: a male and a female component. The insertion of the nail is achieved in a single step; the male and female components are simultaneously introduced into the intramedullary canal and fixed distally and proximally. Simultaneous insertion significantly reduces the implant insertion time and streamlines the surgical technique. Moreover, simultaneous insertion helps prevent friction resistance compared to traditional insertion methods, where the male component may already be bent, making it less receptive to accommodating the female component. Furthermore, the additional rotational stability is crucial during the initial weeks of bone realignment.

### BACKGROUND

Children born with rare bone conditions, such as osteogenesis imperfecta (OI), congenital pseudarthrosis, hypophosphatemia rickets, fibrous dysplasia, exhibit diminished bone quality. The compromised bone quality in these children leads to bowed long bones and frequent fractures. In such cases, intramedullary rodding is recommended to manage the recurrent fractures of long bones and alleviate bone deformities that impede normal functions. Intramedullary rodding entails the insertion of a metallic rod into the medullary canal of the long bone, with the inserted rod being either a Non-Telescoping Nail or a Telescoping Nail.

Non-telescoping nails are inserted to provide support along the entire length of the bone for realignment. In 1959, Sofield and Millar (Sofield H A, 1959) introduced their multiple realignment osteotomies technique, commonly known as the “shish kebab,” utilizing a non-telescopic intramedullary rod. Although these non-telescoping nails enhance bone strength, they do not accommodate the child's growth, and the longitudinal bone surpasses the nail. Consequently, periodic replacements of the non-telescoping nail become necessary after a certain degree of bone growth. Additionally, there is a risk of bone bowing beyond the point where the rod terminates, potentially resulting in a failure of deformity correction. These significant drawbacks prompted the development of telescoping nails, capable of lengthening as the bone grows

The concept of extensible nails was pioneered by Dr. Bailey Robert in 1962 (Dubow H I., 1963) and was commercially available by Zimmer corp. The apparatus comprises two components: an outer hollow nail with a removable threaded T-end and an inner solid rod with a removable T-end. This introduced implant was recognized as the Dubow-Bailey rod. The proposed design facilitated telescoping to accommodate the longitudinal bone's growth. The Dubow-Bailey device was often employed in conjunction with the Sofield fragmentation technique, proving effective in reducing the number and frequency of operations needed for growing children. However, the use of Dubow-Bailey was considered technically demanding, and the complication rate remained high. The most prevalent issues included proximal rod migration and disengagement of the epiphyseal T-piece.

To address the challenges posed by the Dubow-Bailey nail, the Sheffield telescopic rod was introduced in 1980. The Sheffield's nail represented an advancement of the Dubow-Bailey telescoping rod, aiming to alleviate issues related to the T-piece. The Sheffield telescoping rods comprise two components: a female hollow nail and a male solid rod. Both components feature an integrated T-shaped end. The T-end shape is incorporated into the distal and proximal physis. However, the Sheffield rod introduced other complications, including intra-articular, metaphyseal, or extra-cortical rod migration. (Tae-Joon Cho, 2007), (Oliver Birke, 2011)

Both the Dubow-Bailey and the Sheffield Rods were rotationally stable. These devices provided rotary or torsional rotational or torsional stress control. This stability permits retaining the functional use of the corrected extremity. However, the insertion technique of the two telescoping components still required a knee arthrotomy for a femoral rod insertion and both knee and ankle arthrotomies for a tibial rod insertion (Niemann, 1981) (Stockley I, 1989) (Wilkinson J M, 1998)

Other improved versions of the Sheffield telescoping rod existed. One of those improvements can be found in (U.S. Pat. No. 6,524,313 B1, 2003), entitled intramedullary nail system. In this improved version of the Sheffield rod, the two T-end shapes were changed by threaded ends. The simplified surgical technique required the removal of the rotational stability. The nail was commercially named the Fassier-Duval IM, or FD nail. The threaded ends of the FD nail allowed an easier and less invasive insertion technique. Nevertheless, the product was rotationally unstable. The smooth telescoping components are multiplied by insufficient longitudinal bone stability and diminished healing capacity, the equation might be rather unfavorable [8]. If the male rod is already in its desired final position in the epiphysis before the insertion of the female rod, the resistance and friction can sometimes drive the male component into the physis. (Zeitlin L, 2003)

For the FD nail, the male component is cut intraoperative and left protruding. For femoral rodding, intraoperative cutting can be performed but it is not a friendly user process, especially for small children. However, in tibial rodding with FD-rods, a slightly protruding male component can irritate the joint of the knee. For the FD, Birke et al. stated that it can be difficult and sometimes impossible to cut the male rod flush or close to flush with the female component after both components have been inserted (Oliver Birke, 2011). If not gently performed, the disengagement of the male driver can cause the male to disengage from the bone.

Another issue with the FD-rod is the extraction technique, for revision, which requires sophisticated instrumentation. For the smallest size of the FD, the FD extraction system obliges the surgeon to over-ream the weakened growing bone to be able to extract the nail to be revised. For example, when a 3.2 MM implant size is revised, it has to be replaced by a 4.8 MM implant instead of a 4.0 MM. This is due to the size of the Shafts of the FD extraction system. To insert the shaft retriever of the FD system, the surgeon has to ream over the male component. The extraction of the FD female components is challenging for surgeons because the female component does not have any integrated extraction feature

Another version of a telescopic nail was patented under (WO 2016/175729 A1) This nail overcame the main disadvantage of the FD system: The absence of rotational stability. This is provided by a D-Shaped female all along and a male with a negative form. This TSTSAN nail is described as a telescopic nail comprised of a male and female component, with two threaded ends, a D-shaped female component to hinder rotation, that is self-tapping and capable of auto-adjusting during bone growth.

The nail developed by the DScope by PediTst system suffers from a curved shape that does not help to realign properly the bone deformity (U.S. Pat. No. 10,433,886B2, 2019). While it is stable it has the same concept of insertion as the FD nail. In fact, the male is inserted first followed by the female component. Which leads to similar disadvantages as the FD nail. The DScope nail requires also an intraoperative cutting which is unfavorable for tibial rodding and does not help to preserve healthy knees. No data was found concerning the extraction technique for this newly launched system. (U.S. Pat. No. 4,016,874, 1977) describes another telescopic nail design with three-part intramedullary bone setting pin that is telescopically received with two threaded ends and is self-tapping.

On of the major inconvenience with existing telescoping nails is the retrieval or extraction either for revision or after physis closure. The extraction of the male component is sometimes challenging due to bone growth all around in the intramedullary canal.

### SUMMARY

A female component with a tapered head with multiple-lead thread.

A female component with additional fixation features on its head

A female component with a cannulated all along.

A female component with a partial or total D-Shaped cannulation.

A D-Shaped form on the female component can be internally or externally located on the distal or proximal section of the component shaft.

A female component with self-tapping features

A male component with multiple leads threaded head and double-moon-shaped shaft.

The male component has a locking feature distally. The locking feature can be of various forms.

The male component has self-taping features.

The Female component can receive the male allowing translational movement only.

The leads of the male and female components can be 2, 3, 4 or any other combination of pitch and lead to adapt to the bone anatomy.

The male and female assembly has an adjustable length, that can be prepared prior to insertion

Additional objectives, advantages, and novel features will be set forth in the description which follows or will become apparent to those skilled in the art upon examination of the drawings and detailed description which follows.

### BRIEF DESCRIPTION OF THE DRAWINGS

FIG. 1A is an isometric view of the telescopic nail Assembly;

FIG. 1B is a section view of the telescopic Assembly

FIG. 2 is a side view of the female component

FIG. 3 is an isometric view of the male component

FIG. 4 is a detailed view of the Female tapered head

FIG. 5 is a side view of the male component head

Reference characters indicate corresponding elements in the views of the drawings. The titles used in the figures should not be interpreted as limiting the scope of the claims.

### DETAILED DESCRIPTION

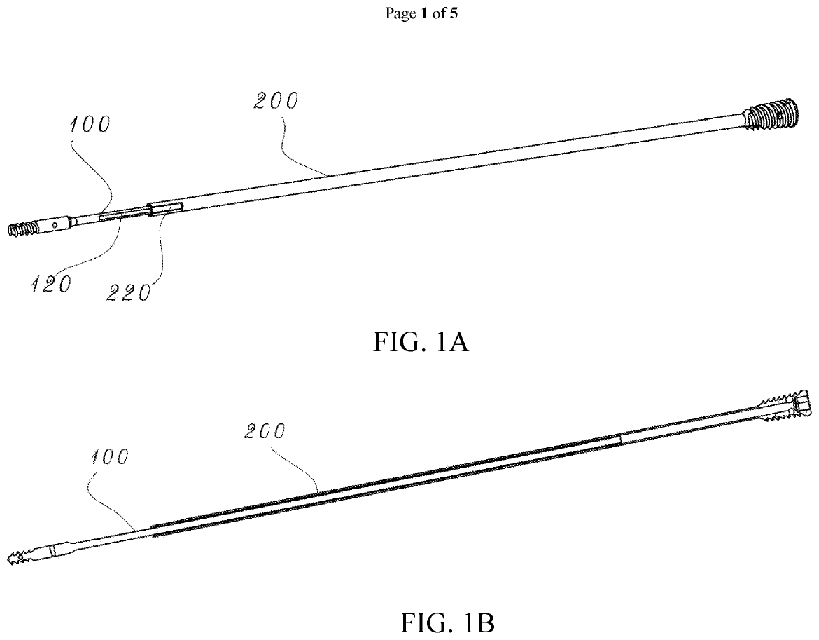

Referring to FIGS. 1A and 1B, the Telescoping Nail assembly is shown in both isometric and sectional view. The isometric view (FIG. 1A) illustrates the locking assembly created by the D-Shape shaft 220 nesting the externally D-Shaped 120 of the female component. This locking allows gliding without rotation during bone growth.

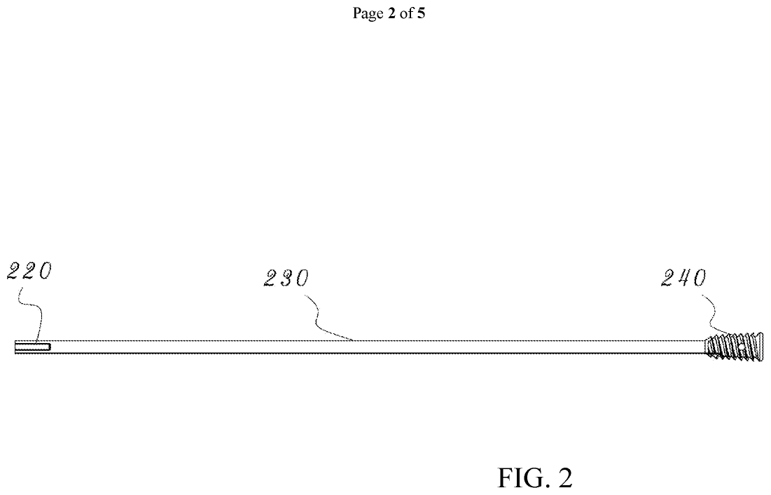

Referring to FIG. 2, a full side view of the female component is shown. The D-Shape 220, the cannulated 230 shafts, and the tapered head 240 are depicted.

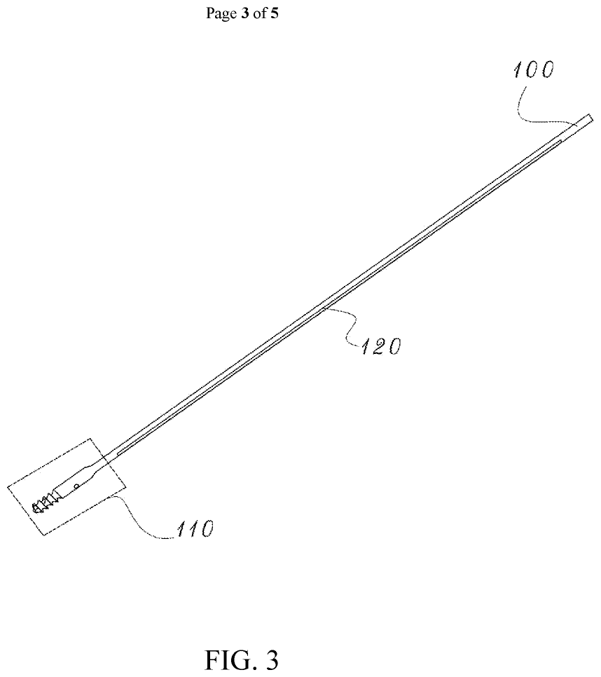

Referring to FIG. 3, an isometric view of the male component. The D-Shape 120 of the male component is shown to be in the mid-shaft region. The D-Shape is not all along the shaft to allow locking between the male and the female. This locking geometry is useful for screw revision, extraction, or removal.

Referring to FIG. 3 is an isometric view of the male component. The head of the male 110 is indicated by its features for fixation, locking, and preservation.

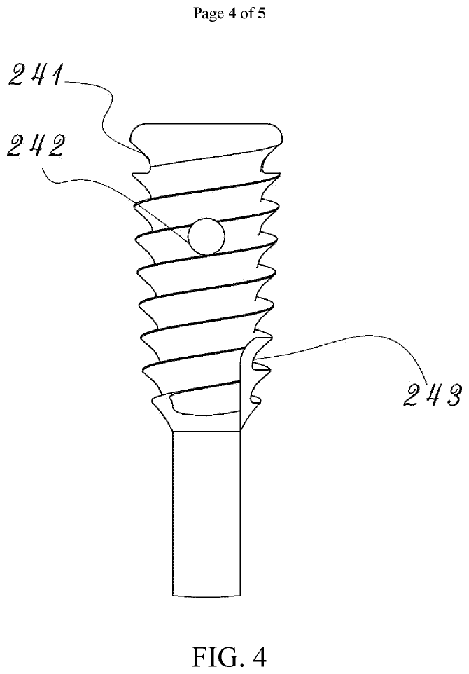

Referring to FIG. 4 is a side view of the female tapered head. The head has a multi-lead thread 241 that is synched with the distal thread on the male head. The thread length is selected as per the child's bone anatomy.

Referring to FIG. 4, Shown is the female head has a locking feature 242 that can be used to secure the female component against positive or negative bone migration. The female head has two self-tapping features 243. The self-tapping features unleash the bone for thread insertion.

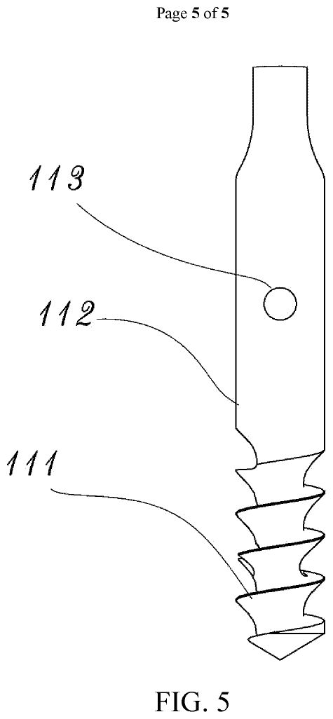

Referring to FIG. 5, is a side view of the male head showing the distally threaded portion with a multi-lead thread 111. That thread is synched with the proximal thread on the female head. The thread length is selected as per the child's bone anatomy.

Referring to FIG. 5, shows the pre-thread shaft of the male component with a diameter larger than the thread major diameter 112. This feature 112 plays an important role in the preservation of the growth plate. This feature 112 acts like a “plug” that blocks cellular migration from the physis to the metaphysis. This blockage is essential for better healing of the growth plate and the preservation of its functionality.

Referring to FIG. 5 shows the locking feature of the male component distally in the metaphysis 113 that can be used to secure the male component against positive or negative bone migration.

While particular embodiments of the invention were illustrated and depicted, various modifications are possible without dissolution of the invention's spirit and scope as will be obvious to those skilled in the art. Such changes and modifications are within the scope and teachings of this invention as defined in the claims appended hereto.

What is claimed is:

1. A telescopic nail assembly of two components a male component nested in a female for the purpose of gliding inside the bone without rotating wherein the female component has an external D-Shaped and the male component has a shaft that is D-Shaped, the male component is distally threaded and lockable and the female component is proximally threaded and lockable, wherein the threads are synched.

2. The telescopic nail assembly of claim 1, wherein the D-Shape of the female component can be located externally partially proximally, or distally.

3. The telescopic nail assembly of claim 1, wherein the external D-Shape of the female component is obtained by a special burnishing or any plastic deformation operation that allows a controlled deformation.

4. The telescopic nail assembly of claim 1, wherein the head of the female component is tapered and has multiple lead threads and additional proximal fixation features.

5. The telescopic nail assembly of claim 1, wherein the head of the male component has multiple lead threads and additional distal fixation features.

6. The telescopic nail assembly of claim 1, wherein the length of the threads on both components proportionally matched in such a way that when the female component head is completely buried in the proximal bone the male component passes through the growth plate in the distal epiphysis.

7. The telescopic nail assembly of claim 1, wherein the female component thread leads can be simple, double, triple quadruple, or any combination of pitch and lead to adapt to the bone anatomy.

8. The telescopic nail assembly of claim 1, wherein the male component thread leads can be simple, double, triple quadruple, or any combination of pitch and lead to adapt to the bone anatomy.

9. The telescopic nail assembly of claim 1, wherein the female head has a proximal hole that offers the possibility of proximal locking.

10. The telescopic nail assembly of claim 1, wherein the female head has two self-tapping features that help to ease the insertion process of the female component in bone.

11. The telescopic nail assembly of claim 1, wherein the shaft of the male is D-Shaped partially on a midshaft portion only.

12. The telescopic nail assembly of claim 1, wherein the distal thread on the male component is smaller than the shaft diameter.

13. The telescopic nail assembly of claim 1, wherein the smooth distal shaft on the male component is greater than the distal thread.

14. The telescopic nail assembly of claim 1, wherein the head of the male component has a distal locking hole.

15. The telescopic nail assembly of claim 1, wherein the head of the male component has a self-tapping feature that eases the insertion of the male component in the bone.

16. A method wherein the telescopic nail assembly of claim 1, wherein the total length of the assembly is adjusted to the child's bone without intra-operative cutting.

17. A method wherein the telescopic nail assembly of claim 1 is inserted simultaneously in the intramedullary canal of the bone.

18. A method wherein the telescopic nail assembly of claim 1 is extracted simultaneously from the intramedullary canal of the bone in case of a hardware removal surgery or for revision surgery.
